(19) United States
(12) Patent Application Publication
(10) Pub. No.: US 2012/0289597 A1
(43) Pub. Date: Nov. 15, 2012

(54) NITRIC OXIDE AMINO ACID ESTERS FOR THE TREATMENT OF CHRONIC PAIN

(75) Inventor: Michael FARBER, Montreal (CA)

(73) Assignee: ORAL DELIVERY TECHNOLOGY LTD., Montreal (CA)

(21) Appl. No.: 13/470,708

(22) Filed: May 14, 2012

Related U.S. Application Data
(63) Continuation of application No. PCT/CA2010/001726, filed on Oct. 28, 2010.
(60) Provisional application No. 61/260,886, filed on Nov. 13, 2009.

(51) Int. Cl. A61K 31/223; A61P 25/04

(52) U.S. Cl. 514/551

(57) ABSTRACT

There is provided a topical composition and a method for treating or alleviating pain in a patient in need thereof. The topical composition contain amino acid ester compounds comprising at least one nitric oxide releasing group and pharmaceutical salts thereof and a topical analgesic compound.

### CROSS-REFERENCE TO RELATED APPLICATIONS

This application claims priority from U.S. Provisional patent application No. 61/260,886 filed Nov. 13, 2010.

### BACKGROUND

(a) Field

The subject matter disclosed generally relates to compositions and methods for the treatment of chronic pain, and more particularly to compositions and methods for treating chronic pain comprising an amino acid ester compound and a topical analgesic compound.

(b) Related Prior Art

Pain is the most common and among the most troubling manifestations of a variety of diseases ranging from arthritis to cancer. A wide variety of analgesics have been employed to relieve or alleviate pain. No single analgesic is uniformly effective and the use of many of these agents is limited by undesirable side effects or substance abuse profiles. Some painful disorders have been particularly resistant to treatment and these include chronic neuropathic pain syndromes such as postherpetic neuralgia and painful diabetic neuropathy, as well as other chronic painful disorders such as painful fibromuscular diseases.

The non-narcotic analgesic acetaminophen and the nonsteroidal anti-inflammatory drugs (NSAIDs) are a heterogeneous group of chemical compounds which have proved very useful in treating many types of common acute pain, such as headache or backache, as well as the chronic pain associated with osteoarthritis.

Nitric oxide (NO) is synthesized from L-Arginine through the action of the enzyme NOs (nitric oxide synthetase), NO is synthesized by different NOs enzymes: nNOs (neuronal NOs), which is present in the cytoplasm of the parasympathetic nerves, and eNOs (endothelial NOs), found in the endothelium of the blood vessels and trabecular tissue, which mainly seems to bond to the cell membranes. Numerous experiments have demonstrated that stimulation of the parasympathetic nerves leads to the release of NO as a result of direct action by the nerve endings (reaction catalyzed by nNOs) and indirect action resulting from the effect of Ach, released by the parasympathetic nerves, on the vascular endothelium, with stimulation of eNos. Nitric oxide has been implicated in the mechanisms of pain generation and supplementation of NO has been implicated in alleviation of some form of chronic pain. For example U.S. Pat. Application No. 2005/0095278 discloses that NO, through the use of topically applied nitroglycerin, is an efficient treatment for relief of pain caused by nocturnal muscle cramps.

It is thus desirable to provide a composition and method for the treatment of chronic pain which contains an alternative compound than nitroglycerin, and does not require any special operational procedures other than the application of a composition onto the painful tissue.

### SUMMARY

In a first embodiment there is disclosed a topical composition comprising

an effective amount of a compound of formula (I):

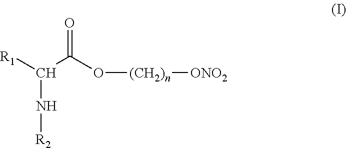

wherein n 1 to 10;
wherein R₁ is chosen an amino acid side chain group (D or L configuration),
wherein R₂ is a hydrogen atom, or an amino acid (D or L configuration) forming a peptide bond, or any pharmaceutically acceptable salts thereof; and

an effective amount of a topical analgesic compound,

in association with a pharmaceutically acceptable topical carrier.

The compound of formula (1) may be (2-nitrooxy)-2-ethylamino-3-methylbutanoate:

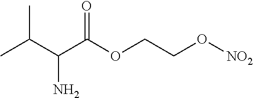

or any pharmaceutically acceptable salts thereof.

The compound of formula (1) may be valine butylene glycol nitrate:

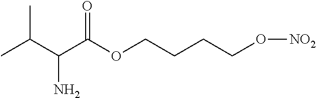

or any pharmaceutically acceptable salts thereof.

The compound of formula (1) may be 2′-nitrooxyethyl 2-amino-pentanoate.

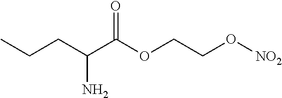

or any pharmaceutically acceptable salts thereof.

The compound of formula (I) may be 4′-nitrooxybutyl 2-amino-pentanoate:

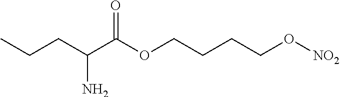

or any pharmaceutically acceptable salts thereof.

The compound of formula (I) may be:

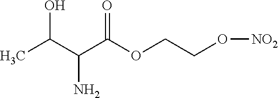

or any pharmaceutically acceptable salts thereof.

The R₂ may be a hydrogen atom.

The R₁ may be chosen from:

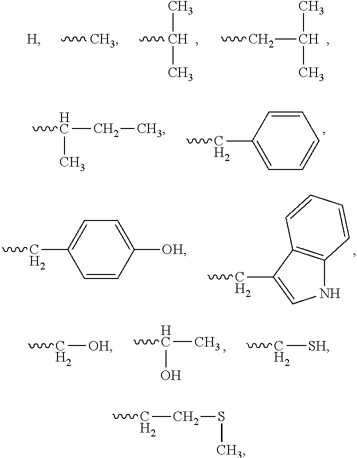

proline side chain,

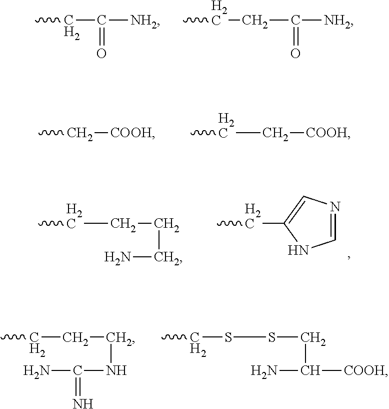

hydroxyproline side chain,

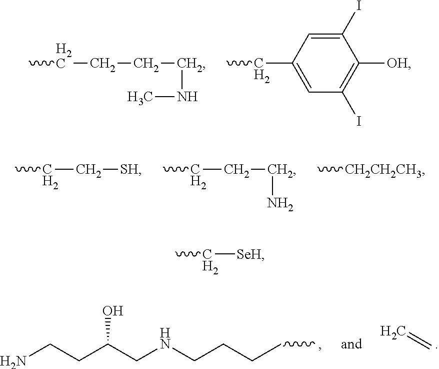

The R₂ is an amino acid of formula (II) (D or L configuration) and derivatives thereof, forming a peptide bond:

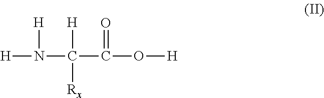

wherein Rₓ is chosen from:

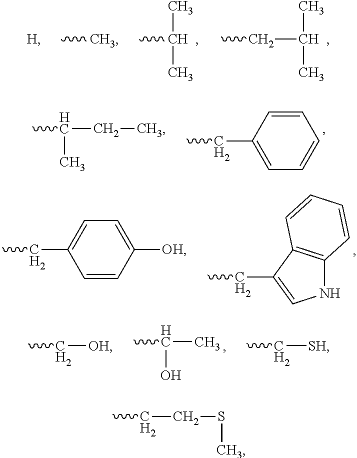

proline side chain,

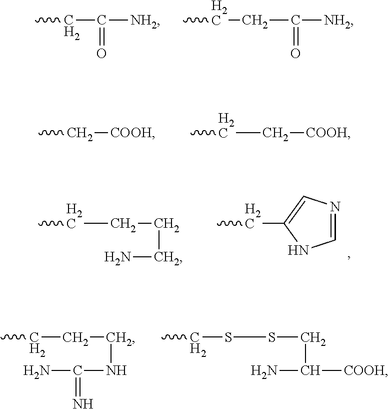

hydroxyproline side chain,

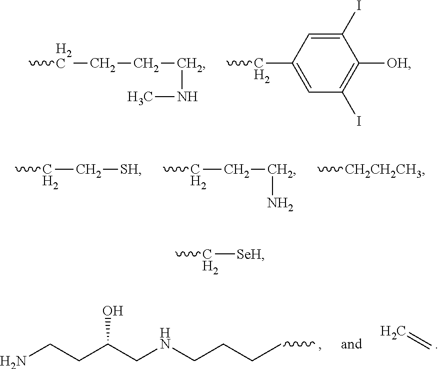

The topical analgesic compound may be chosen from a capsaicinoid, resiniferatoxin, cinnamaldehyde, menthol, eucalyptol, camphor, and norcamphor.

The capsaicinoid may be chosen from capsaicin, dihydrocapsaicin, nordihydrocapsaicin, homodihydro capsaicin, homocapsaicin, and nonivamide.

The pharmaceutically acceptable topical carrier may be chosen from a water base or an oil base carrier.

The anti-oxidants may be selected from glutathione, vitamin C, alpha lipoic acid, beta-carotene, alpha-tocopherols, lutein and combinations thereof.

The moisturizer may be selected from stearic acid, myrestyl alcohol, white petrolatum, glycerin, lanolin, hydrogenated polydecene, cetearyl alcohol and combinations thereof.

The humectant may be selected from glyceryl triacetate, sorbitol, quillaia, urea, glycerin, lactic acid, aloe vera, propylene glycol and combinations thereof.

The emollient may be selected from butyrospermum parkii oil, licithin, olive oil, glyceryl stearate, stearyl alcohol, cetyl alcohol, behenyl alcohol, limnanthes alba seed oil, palmitic acid and combinations thereof.

The healing promoting agent may be selected from collagen hydrolysate, aldioxa, hyaluronic acid, elastin, ascorbyl palmitate, and combinations thereof.

The dermal circulation enhancer may be gingko biloba, ginger, ethyl alcohol, arginine, cayenne and combinations thereof.

The vitamin may be selected from vitamin A, vitamin E, vitamin C, vitamin D and combinations thereof.

The mineral may be selected from zinc, sodium, potassium, selinium, manganese, copper, calcium and combinations thereof.

The emulsifier may be selected from sodium lauryl sulfate, trideceth-6, pluronic acid F-127, polyacrylate sodium, triethanolamin, hydroxyethylcetearamidopropyl dimonium chloride and combinations thereof.

The composition may comprise a lubricant, and the lubricant may be glycerol, sorbitol, a water soluble cellulose, a polysorbate, a carborner, a polyethylene glycol (PEG), a polyethylene, and a thickening agent.

The water soluble cellulose may be selected from modified starch, methylcellulose, hydroxypropyl cellulose, hydroxypropyl methylcellulose, methocel® MC, carboxymethyl cellulose, ethyl cellulose, hydroxyl ethyl cellulose, and any combination thereof.

The polysorbate may be selected from polyoxyethylene (20) sorbitan monolaurate (polysorbate 20), polyoxyethylene (20) sorbitan monopalmitate (polysorbate 40), polyoxyethylene (20) sorbitan monostearate (polysorban 60), polyoxyethylene (20) sorbitan tristrearate (polysorban 65), and polyoxyethylene (20) sorbitan monooleate (polysorban 80), and any combination thereof.

The carbomer may be a carbopol® polymer chosen from carbopol® polymer 71G NE, carbopol® polymer 971P NE, carbopol® polymer 974P NE, carbopol® polymer 980 NE, carbopol® polymer 981 NE, carbopol® polymer 5984 EP and carbopol® polymer Ultrez 10 NF, and any combination thereof.

The polyethylene glycol (PEG) may be selected from PEG 200, PEG 200E, PEG 300, PEG 300E, PEG 400, PEG 400E, PEG 600 and PEG 600E, and any combination thereof.

The thickening agent may be selected from alginic acid, sodium alginate, potassium alginate, ammonium alginate, calcium alginate, agar, carrageenan, locust bean gum, xanthan gum, pectin, and gelatin, and any combination thereof.

The composition may be comprising at least one antiseptic agent.

The antiseptic agent may selected from chlorhexidine gluconate, glucono delta-lactone, a paraben compound, benzoic acid, imidazolidinyl urea, a quaternary ammonium compound, and Octenidine dihydrochloride.

The composition may be comprising a preservative agent.

The preservative agent may be selected from EDTA, EGTA, hydroxytoluene butoxide, hydroxyanisol butoxide, sodium hydroxide, calcium propionate, sodium nitrate, sodium nitrite, sulfur dioxide, sodium bisulfite, and potassium hydrogen sulfite.

The composition may be comprising an absorption enhancer.

The absorption enhancer may be triglycerides of coconut oil, isopropyl palmitate, isopropyl myristate, laurocapram, glycerol, propylene glycol and derivatives thereof.

The composition may be a transdermally absorbed composition.

In accordance with a second embodiment, there iS disclosed a method of reducing or alleviating pain in a patient which comprises:
(a) topically treating said patient with the composition of the present invention.

in another embodiment, there is disclosed a method of reducing or alleviating pain in a patient which comprises:
(a) topically treating said patient with a composition comprising:

an effective amount of a compound of formula (1):

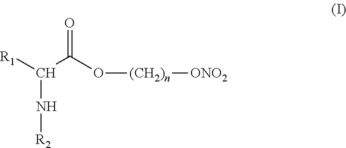

wherein n =1 to 10;
wherein R₁ is chosen an amino acid side chain group (D or L configuration),
wherein R₂ is a hydrogen atom, or an amino acid (D or L configuration) forming a peptide bond, or any pharmaceutically acceptable salts thereof;
in association with a pharmaceutically acceptable topical carrier.

The compound of formula (I) may be (2-nitrooxy)-2-ethylamino-3-methylbutanoate:

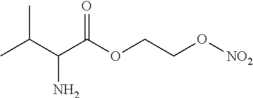

or any pharmaceutically acceptable salts thereof.

The compound of formula (1) may be valine butylene glycol nitrate:

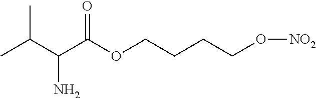

or any pharmaceutically acceptable salts thereof.

The compound of formula (I) may be 2′-nitrooxyethyl 2-amino-pentanoate:

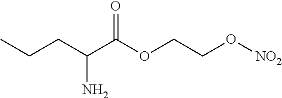

or any pharmaceutically acceptable salts thereof.

The compound of formula (I) may be 4′-nitrooxybutyl 2-amino-pentanoate:

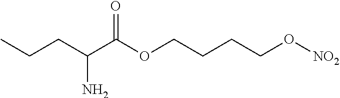

or any pharmaceutically acceptable salts thereof.

The compound of formula (I) may be:

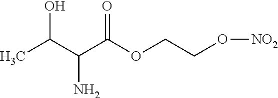

or any pharmaceutically acceptable salts thereof.

The R₂ may be a hydrogen atom.

The R₁ may be chosen from:

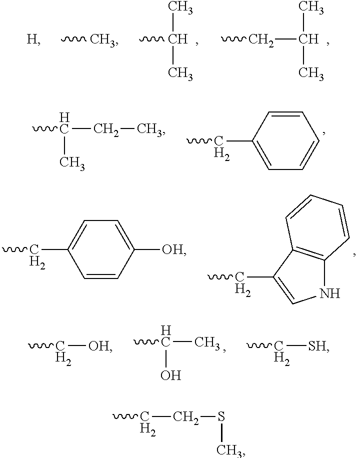

proline side chain,

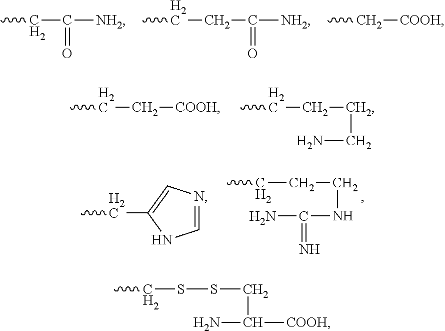

hydroxyproline side chain,

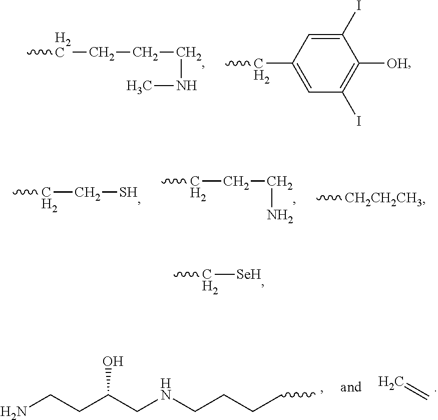

The R₂ may be an amino acid of formula (II) (D or L configuration) forming a peptide bond:

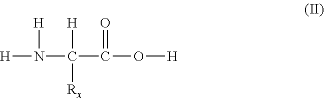

The Rₓ may be chosen from:

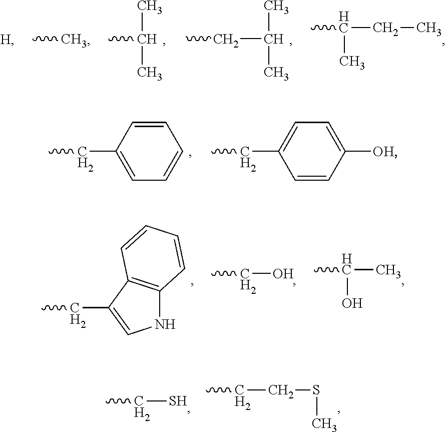

proline side chain,

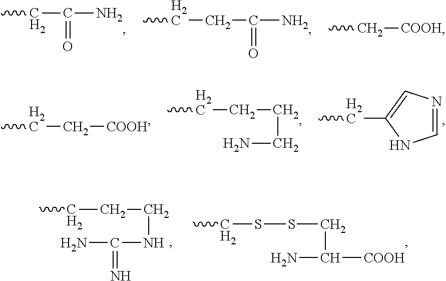

hydroxyproline side chain,

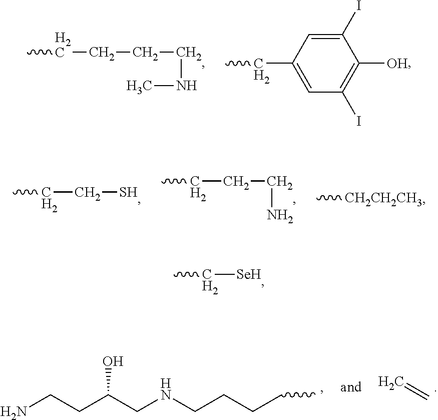

The pharmaceutically acceptable topical carrier may be chosen from a cream, a gel and a lotion.

The topical composition may contain an anti-oxidants selected from glutathione, vitamin C, alpha lipoic acid, beta-carotene, alpha-tocopherols, lutein and combinations thereof.

The topical composition may contain a moisturizer selected from stearic acid, myrestyl alcohol, white petrolatum, glycerin, lanolin, hydrogenated polydecene, cetearyl alcohol and combinations thereof.

The topical composition may comprise a humectant selected from glyceryl triacetate, sorbitol, quillaia, urea, glycerin, lactic acid, aloe vera, propylene glycol and combinations thereof.

The topical composition may comprise an emollient selected from butyrospermum parkii oil, licithin, olive oil, glyceryl stearate, stearyl alcohol, cetyl alcohol, behenyl alcohol, limnanthes alba seed oil, palmitic acid and combinations thereof.

The topical composition may comprise a healing promoting agent selected from collagen hydrolysate, aldioxa, hyaluronic acid, elastin, ascorbyl palmitate and combinations thereof.

The topical composition may comprise a dermal circulation enhancer selected from gingko biloba, ginger, ethyl alcohol, arginine, cayenne and combinations thereof.

The topical composition may comprise a vitamin selected from vitamin A, biotin, vitamin E, vitamin C, vitamin D and combinations thereof.

The topical composition may comprise a mineral selected from zinc, sodium, potassium, selinium, manganese, copper, calcium and combinations thereof.

The topical composition may comprise an emulsifier selected from sodium lauryl sulfate, trideceth-6, pluronic acid F-127, polyacrylate sodium, triethanolamin, hydroxyethylcetearamidopropyl dimonium chloride and combinations thereof.

The topical composition may comprise a lubricant.

The lubricant may be chosen from glycerol, sorbitol, a water soluble cellulose, a polysorbate, a carbomer, a polyethylene glycol (PEG), a polyethylene, and a thickening agent.

The water soluble cellulose may be chosen from modified starch, methylcellulose, hydroxypropyl cellulose, hydroxypropyl methylcellulose, methocel® MC, carboxymethyl cellulose, ethyl cellulose, hydroxyl ethyl cellulose, and any combination thereof.

The polysorbate may be chosen from polyoxyethylene (20) sorbitan monolaurate (polysorbate 20), polyoxyethylene (20) sorbitan monopalmitate (polysorbate 40), polyoxyethylene (20) sorbitan monostearate (polysorban 60), polyoxyethylene (20) sorbitan tristrearate (polysorban 65), and polyoxyethylene (20) sorbitan monooleate (polysorban 80), and any combination thereof.

The carbomer may be a carbopol® polymer chosen from carbopol® polymer 71G NF, carbopol® polymer 971P NF, carbopol® polymer 974P NF, carbopol® polymer 980 NF, carbopol® polymer 981 NF, carbopol® polymer 5984 EP and carbopol® polymer Ultrez 10 NF, and any combination thereof.

The polyethylene glycol (PEG) may be chosen from PEG 200, PEG 200E, PEG 300, PEG 300E, PEG 400, PEG 400E, PEG 600 and PEG 600E, and any combination thereof.

The thickening agent may be chosen from alginic acid, sodium alginate, potassium alginate, ammonium alginate, calcium alginate, agar, carrageenan, locust bean gum, xanthan gum, pectin, and gelatin, and any combination thereof.

The composition may be a transdermally absorbed composition.

The composition may comprise at least one antiseptic agent.

The antiseptic agent may be selected from chlorhexidine gluconate, glucono delta-lactone, a paraben compound, benzoic acid, imidazolidinyl urea, a quaternary ammonium compound, and Octenidine dihydrochloride.

The composition may further comprises a preservative agent chosen from EDTA, EGTA, hydroxytoluene butoxide, hydroxyanisol butoxide, sodium hydroxide, calcium propionate, sodium nitrate, sodium nitrite, sulfur dioxide, sodium bisulfite, benzoic acid, caprylyl glycol, Diazolidinyl urea, Phenoxyethanol, Dehydroacetic acid, Iodopropynylbutylcarbamate, Sorbic acid, Isopropyl-paraben, Isobutyl-paraben, Butyl-paraben and potassium hydrogen sulfite.

The composition may be comprising an absorption enhancer,

The absorption enhancer may be chosen from triglycerides of coconut oil, isopropyl palmitate, isopropyl myristate, laurocapram, glycerol, propylene glycol and derivatives thereof.

The patient may have a normotensive blood pressure, a hypertensive blood pressure, or a hypotensive blood pressure.

The blood pressure may be a normotensive blood pressure or a hypotensive blood pressure, said topically treating said patient results in a stable blood pressure.

The blood pressure may be a hypertensive blood pressure, said topically treating said patient results in a decreased blood pressure.

The decreased blood pressure may be a normotensive blood pressure.

In another embodiment, there is disclosed a use of a compound of formula (I) for reducing or alleviating pain:

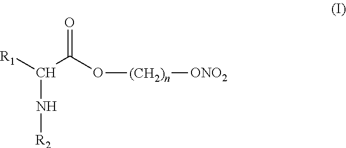

wherein n=1 to 10;

wherein R₁ is an amino acid side chain group (D or L configuration),

wherein R₂ is a hydrogen atom, or an amino acid (D or L configuration) forming a peptide bond,

or any pharmaceutically acceptable salts thereof.

The R₁ may be chosen from:

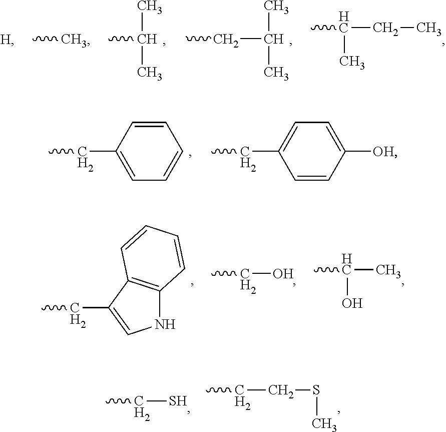

proline side chain,

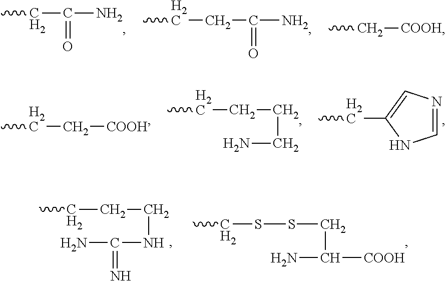

hydroxyproline side chain,

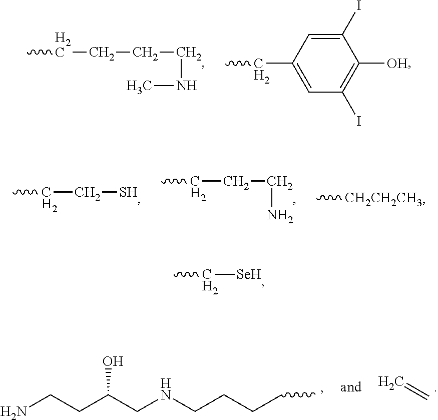

The R₂ may be an amino acid of formula (II) (D or L configuration) farming a peptide bond:

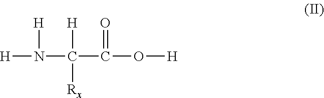

wherein Rₓ is chosen from

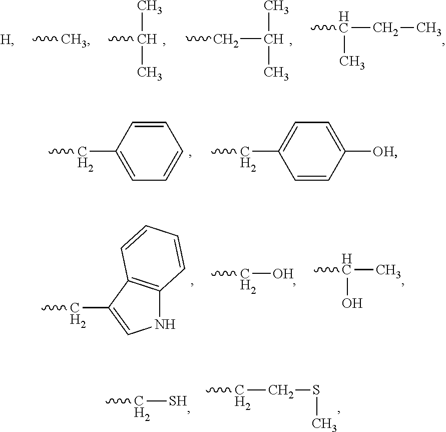

praline side chain,

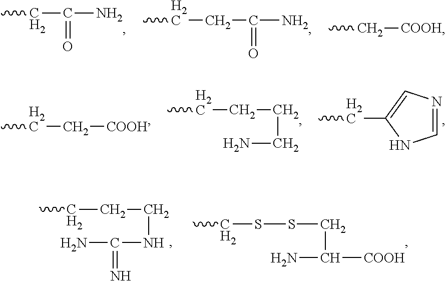

hydroxyproline side chain,

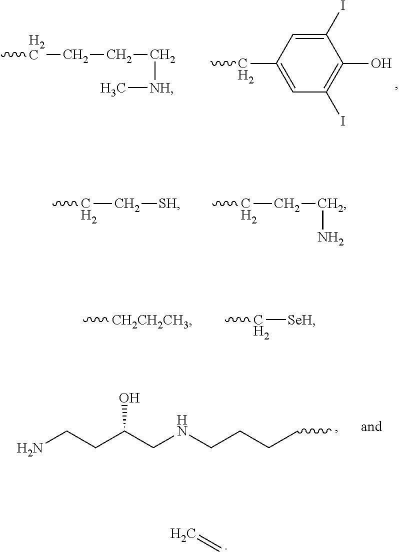

The compound of formula (1) may be (2-nitrooxy)-2-ethylamino-3-methylbutanoate.

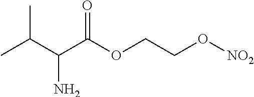

or any pharmaceutically acceptable salts thereof.

The compound of formula (1) may be valine butylene glycol nitrate:

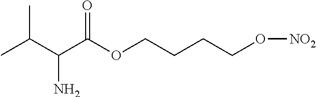

or any pharmaceutically acceptable salts thereof.

The compound of formula (I) may be 2′-nitrooxyethyl 2-amino-pentanoate:

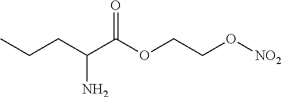

or any pharmaceutically acceptable salts thereof.

The compound of formula (ay be 4′-nitrooxybutyl 2-amino-pentanoate:

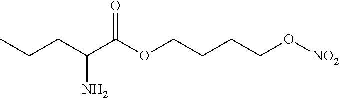

or any pharmaceutically acceptable salts thereof.

The compound of formula (I) may be:

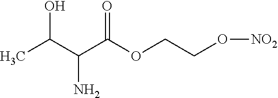

or any pharmaceutically acceptable salts thereof.

The R₂ is a hydrogen atom

The following terms are defined below

The term “Chronic pain” is intended to mean nocturnal muscle cramps, arthritis, rheumatoid arthritis, cancer, chronic neuropathic pain syndromes such as postherpetic neuralgia and painful diabetic neuropathy, fibromuscular diseases, tension, headache, backache, osteoarthritis, migraine, tension headache, anal fissure pain, and Raynaud's Phenomenon.

The term “Amino acid ester compound” is intended to mean the condensation product of an amino acid with mononitrated alkane cu alkene diol. As will be evident to those familiar to the art, the condensation reaction could also involve, but not limited to, dipeptides or tripeptides, nitrated alcohols containing aliphatic, alkyl or aromatic moieties, as well as other nitric oxide groups attached to the alkane or alkene dials. Amino acid or dipeptide reactions are preferred as well as the condensation reaction with short chain mononitrated alkane diols such as 1,3 propanediol or 1,4 butanediol.

The expression “Therapeutically effective amount” is intended to mean the amount of the compound and/or composition that is effective to achieve its intended purpose.

The expression “Transdermally absorbed” is intended to mean the delivery of a compound by passage through the skin and into the blood stream.

The term “Transmucosal” is intended to mean the delivery of a compound by passage of the compound through the mucosal tissue and into the blood stream.

The terms “Carriers” or “vehicles” are intended to mean carrier materials suitable for compound administration and include any such material known in the art such as, for example, any liquid, lotion, gel, solvent, liquid diluent, solubilizer, or the like, which is non-toxic and which does not interact with any components of the composition in a deleterious manner.

The term “Nitric oxide adduct” or “NO adduct” is intended to mean compounds and functional groups which, under physiological conditions, can donate, release and/or directly or indirectly transfer any of the three redox forms of nitrogen monoxide (NO⁺, NO⁻, NO^{∘}), such that the biological activity of the nitrogen monoxide species is expressed at the intended site of action.

The term “Nitric oxide releasing” or “nitric oxide donating” is intended to mean methods of donating, releasing and/or directly or indirectly transferring any of the three redox forms of nitrogen monoxide (NO+, NO−, NO^{∘}), such that the biological activity of the nitrogen monoxide species is expressed at the intended site of action.

The term “Nitric oxide donor” or “NO donor” is intended to mean compounds that donate, release and/or directly or indirectly transfer a nitrogen monoxide species, and/or stimulate the endogenous production of nitric oxide or endothelium-derived relaxing factor (EDRF) in vivo and/or elevate endogenous levels of nitric oxide or EDRF in vivo and/or are oxidized to produce nitric oxide and/or are substrates for nitric oxide synthase and/or cytochrome P450. “NO donor” also includes compounds that are precursors of L-arginine, inhibitors of the enzyme arginase and nitric oxide mediators.

The term “pharmaceutical acceptable carrier” is intended to mean a preservative solution, a saline solution, an isotonic (about 0.9%) saline solution, or about a 5% albumin solution, suspension, sterile water, phosphate buffered saline, and the like. Other buffering agents, dispersing agents, and inert non-toxic substances suitable for delivery to a patient may be included in the compositions of the present invention. The compositions may be solutions, suspensions or any appropriate formulation Suitable for administration, and are typically sterile and free of undesirable particulate matter. The compositions may be sterilized by conventional sterilization techniques.

The term “lubricant” is intended to mean a substance (often a liquid) introduced between two moving surfaces to reduce the friction between them, hydrate the surface as well as reducing wear of the body parts.

The term “pain” in intended to mean physical pain causing an unpleasant feeling and is commonly associated to a headache, a wound, an injury or other forms of bodily suffering. It is normally associated with acute discomfort, Pain May be chronic (persistent, recurring) or acute,

Features and advantages of the subject matter hereof will become more apparent in light of the following detailed description of selected embodiments, as illustrated in the accompanying figures. As will be realized, the subject matter disclosed and claimed is capable of modifications in various respects, all Without departing from the scope of the claims. Accordingly, the drawings and the description are to be regarded as illustrative in nature, and not as restrictive and the full scope of the subject matter is set forth in the claims.

### DETAILED DESCRIPTION OF THE PREFERRED EMBODIMENTS

In a first embodiment there is disclosed a topical composition containing a nitric oxide amino acid ester compound, and a topical analgesic compound, in association with a pharmaceutically acceptable topical carrier.

In another embodiment there is disclosed a method of reducing or alleviating pain in a patient by topically treating said patient with a composition containing a nitric oxide amino acid ester compound and a topical analgesic compound, in association with a pharmaceutically acceptable topical carrier.

In yet another embodiment, there is disclosed a method of reducing or alleviating pain in a patient by topically treating said patient with a composition containing a nitric oxide amino acid ester compound in association with a pharmaceutically acceptable topical carrier.

The composition of the present invention contains vasoactive amino acid ester compounds. The nitric oxide amino acid ester compounds of the present invention possess many of the required characteristics necessary to fulfill the role of a primary boosting of NO levels. The compounds easily dissociate in water into the amino acid derivative and associated ion forming the pharmaceutical salt. The compounds of the present invention are extremely stable in the form of the salts, and thus possess long shelf lives and stability.

The nitric oxide releasing groups of the compounds of the present invention are preferably nitro groups (i.e. NO₂), nitroso groups (i.e. NO) and/or heterocyclic nitric oxide donor groups that are linked to the amino acid ester compounds through one or more sites such as oxygen (hydroxyl condensation), sulfur (sulfhydryl condensation) and/or nitrogen. The heterocyclic nitric oxide donor groups are preferably furoxans, sydnonimines, oxatriazole-5-ones and/or oxatriazoie-5-imines.

The preferred compound of the present invention is the valine derivative of the nitric oxide amino acid ester of the present invention. The most preferred compounds are known as valine nitrooxy ethyl ester (or valine ethylene glycol nitrate), valine nitrooxy butyl ester (or valine butylene glycol nitrate), or any pharmaceutically acceptable salts thereof, which possess many of the required characteristics necessary to fulfill the role of boosting NO levels. The compound easily dissociates in water into the valine derivative valine ethylene or butylene glycol nitrate and the salt forming acid. The compounds are extremely stable in the form of the salt and thus possesses a long shelf life. It has been observed that the preferred compounds of the present invention do not cause hypotension in normotensive or hypotensive individuals. Therefore, upon administration of the preferred compounds of the present invention, an hypertensive individual will experience the vasodilatory effect caused by the preferred compounds, which will result in a decrease in blood pressure. The decrease in blood pressure may be up to a normotensive blood pressure. Individuals with normal blood pressure will not experience the vasodilatory effect caused by the preferred compounds, and their blood pressure will remain stable (unchanged). Individuals with lower than normal blood pressure (hypotensive) will not experience a further drop in blood pressure and their blood pressure will remain stable (unchanged). Furthermore, the preferred compounds of the present invention have half-life of approximately 5 hours. Preferably, a therapeutically effective amount of the compounds of the present invention are administered. Therapeutically effective amounts include but are not limited to 0.5 to 30 mg of the compound of the present invention. Preferably, therapeutically effective amounts include 1 to 15 mg, 0.5 to 5 mg, 1 to 5 mg, 5 to 10 mg, 10 to 15 mg, 1 to 15 mg, 1 to 30 mg, 5 to 20 mg, 5 to 15 mg, 5 to 30 mg, 10 to 20 mg, 10 to 30 mg and 15 to 30 mg.

The compounds and compositions of the invention can be formulated as pharmaceutically acceptable salt forms. Pharmaceutically acceptable salts include, for example, alkali metal salts and addition salts of free acids or free bases. The nature of the salt is not critical, provided that it is pharmaceutically-acceptable. Suitable pharmaceutically-acceptable acid addition salts may be prepared from an inorganic acid or from an organic acid. Examples of such inorganic acids include, but are not limited to, hydrochloric, hydrobromic, hydroiodic, nitric, carbonic, sulfuric and phosphoric acid and the like. Appropriate organic acids include, but are not limited to, aliphatic, cycloaliphatic, aromatic, heterocyclic, carboxylic and sulfonic classes of organic acids, such as, for example, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, β-hydroxybutyric, cyclohexylaminosulfonic, galactaric and galacturonic acid and the like. Suitable pharmaceutically-acceptable base addition salts include, but are not limited to, metallic salts made from aluminum, calcium, lithium, magnesium, potassium, sodium and zinc or organic salts made from primary, secondary and tertiary amines, cyclic amines, N, N′-dibenzylethylenediamine, chloroprocaine, choline, diethanolamine, ethylenediamine, meglumine (N-methylgiucamine) and procaine and the like. All of these salts may be prepared by conventional means from the corresponding compound by reacting, for example, the appropriate acid or base with the compound. In one embodiment, the pharmaceutically acceptable salts of the compounds of the invention include the nitrate salts. In another embodiment, the pharmaceutically acceptable salts of the compounds of the invention are heterocyclic compounds such as, furoxan, a sydnonimine, oxatriazole-5-one and/or an oxatriazole-5-imine

The compounds of the present invention, because of the small size of the molecule, can be other choices of linkages and/or amino acids or their derivatives. For example, as alternatives to the above choices, propyl, butyl, or longer chains may be linked to any amino acid. Salts such as chloride or hydrochloride salts may be used. Other amino acid derivatives may also be chosen. Derivatives of the base amino acids whether they are in the L or D configuration of these amino acids can be chosen. Non standard amino acids, or synthetic derivative of standard and non-standard amino acids may be elected, such as those containing acetyl groups attached to the amide of the molecule or nor derivatives of the amino acids, when such derivatives can be achieved.

The amino acid esters compounds may be based on natural, non-standard or even modified amino acids, with the basic structure as depicted below, where the Rₓ represents the side chain of the amino acid (wherein Rₓ may be R₁, R₂ or R₃, as applicable to the specific molecule described herein):

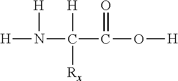

### Basic Amino Acid Structure

Natural Amino Acids

| | Originating | | |
|---|---|---|---|
| N° | Amino acid | Formula | Rₓ = R₁ or R₂ or R₃ |
| 1 | Glycine | H | —H |
| 2 | Alanine | CH₃ | 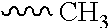 |
| 3 | Valine* | CH(CH₃)₂ | 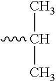 |
| 4 | Leucine* | CH₂CH(CH₃)₂ | 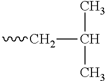 |
| 5 | Isoleucine | CH(CH₃)CH₂CH₅ | 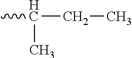 |
| 6 | Phenylalanine* | CH₂C₆H₅ | 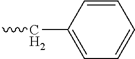 |
| 7 | Tyrosine | CH₂C₆H₄OH | 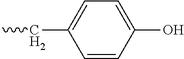 |
| 8 | Tryptophane* | C₉H₈N | 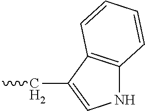 |
| 9 | Serine | CH₂OH | 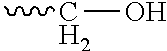 |
| 10 | Threonine* | CH(OH)CH₃ | 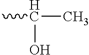 |
| 11 | Cysteine | CH₂SH | 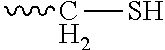 |
| 12 | Methionine* | CH₂CH₂SCH₃ | 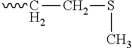 |
| 13 | Proline | C₅H₉NO₂ | 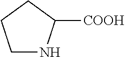 |
| 14 | Asparagine | CH₂COCH₂ | 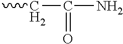 |
| 15 | Glutamine | CH₂CH₂CONH₂ | 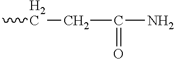 |
| 16 | Aspartic acid | CH₂COOH | 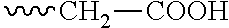 |
| 17 | Glutamic acid | CH₂CH₂COOH | 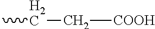 |
| 18 | Lysine* | CH₂CH₂CH₂CH₂NH₂ | 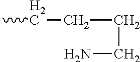 |
| 19 | Histidine* | CH₃C₃N₂H₃ | 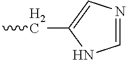 |
| 20 | Arginine* | (CH₂)₃CN₃H₄ | 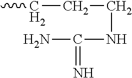 |

*essential amino acids

Modified Amino Acids

| | Originating | | |
|---|---|---|---|
| N° | Amino acid | Formula | Rₓ = R₁ or R₂ or R₃ |
| A | Cystine | CH₂S₂CH₂CHNH₂COOH | 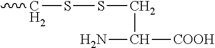 |
| B | Hydroxyproline | C₅H₉NO₃ | 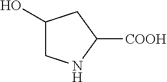 |
| C | ε-N-methyllysine | CH₂CH₂CH₂CH₂NHCH₃ | 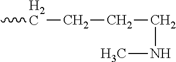 |
| D | diiodotyrosine | CH₂C₆H₂I₂OH | 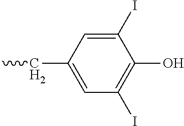 |
| E | homocysteine | CH₂CH₂SH | 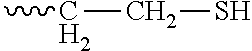 |
| F | ornithine | CH₂CH₂CH₂NH₂ | 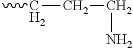 |
| G | Norvaline | CH₂—CH₂—CH₃ | 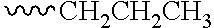 |
| H | selenocysteine | CH₂—SeH | 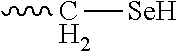 |
| I | Hypusine | CH₂CH₂CH₂CH₂NHCH₂CH(OH)CH₂CH₂NH₂ | 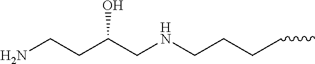 |
| J | Dehydroalanine | CH₂ | 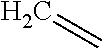 |

The nitric oxide amino acid ester compounds of the present invention are not limited to a single amino acid molecule. The compounds of the present invention may be dipeptide or even tripeptide molecules, with the general formula depicted below and where Rₓ and R_{y} independently are any of the amino acid side chains described herein.

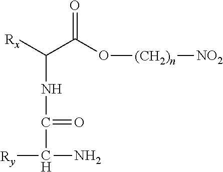

The composition containing a compound as defined in the present invention may include a wide variety of additional components, including, for example, one or more of gases, gaseous precursors, liquids, oils, stabilizing materials, pharmaceutical acceptable carriers, photoactive agents.

The invention provides methods for boosting NO levels for the treatment of certain types of physical pain.

The composition of the present invention may contain a topical analgesic compound to cause a sensation of warmth, a sensation of coolness or both. Such compounds include capsaicinoid compounds, such as capsaicin, dihydrocapsaicin, nordihydrocapsaicin, homodihydro capsaicin, homocapsaicin, and nonivamide. Compounds of this family are the active component of chili peppers, which are plants belonging to the genus Capsicum.

The topical analgesic also include resiniferatoxin, which is a naturally occurring, ultrapotent capsaicin analog that activates the vanilloid receptor in a subpopulation of primary afferent sensory neurons involved in nociception (the transmission of physiological pain).

Also included is cinnamaldehyde, the primary organic compound in cinnamon, menthol, the primary organic compound found in peppermint or other mint oils, eucalyptol (aka limonene oxide, cineol, cineole), which is obtained from plants of the eucalyptus genus, camphor, extracted from the camphor laurel and norcamphor, where three hydrogens have replaced the methyl groups of camphor.

These compounds generate characteristic “burning” or “cold” sensations when put into contact with the skin, which comes from the excitation of nociceptive neurons located thereon. Continued exposure of these neurons to the compounds causes a decrease in the sensitivity of the receptors found on the neurons, which accounts for the analgesic effect of the topical applications of these compounds. Therefore, a patient will initially experience a burning and/or cold sensations that May be difficult to tolerate, and with continued exposure, the patient will usually obtain relief from the desensitizing of the nociceptive neuron.

The composition of the present invention is preferably prepared in the form of a cream, a gel or a lotion. The cream form of the present invention may be typical moisturizing creams formulations that are well known in the art, into which the active ingredients of the present invention are incorporated.

Cream compositions according to the present invention may contain ingredients such as anti-oxidants, moisturizers, humectants, emollients, healing promoting agents and dermal circulation enhancers, vitamins, minerals, emulsifiers, and preservatives.

### Anti-Oxidants

Anti-oxidants include glutathione, vitamin C, alpha lipoic acid, beta-carotene, alpha-tocopherols, lutein and combinations thereof.

Moisturizer

Moisturizers include of stearic acid, myrestyl alcohol, white petrolatum, glycerin, lanolin, hydrogenated polydecene, cetearyl alcohol and combinations thereof.

Humectant

Humectants include glyceryl triacetate, sorbitol, quillaia, urea, glycerin, lactic acid, aloe vera, propylene glycol and combinations thereof.

Emolient

The emollients include butyrospermum parkii oil, licithin, olive oil, glyceryl stearate, stearyl alcohol, cetyl alcohol, behenyl alcohol, limnanthes alba seed oil, palmitic acid and combinations thereof.

Healing Promoting Agents

The healing promoting agents include collagen hydrolysate, aldioxa, hyaluronic acid, elastin, ascorbyl palmitate and combinations thereof.

Dermal Circulation Enhancers

The dermal circulation enhancers include ginger, ethyl alcohol, arginine, cayenne and combinations thereof.

Vitamins

The vitamins include vitamin A, biotin, vitamin E, vitamin C, vitamin D and combinations thereof.

Minerals

The minerals include zinc, sodium, potassium, selinium, manganese, copper, calcium and combinations thereof.

Emulsifiers

The emulsifiers include sodium lauryl sulfate, trideceth-6, pluronic acid F-127, polyacrylate sodium, triethanolamin, hydroxyethylcetearamidopropyl dimonium chloride and combinations thereof.

Keratolytic Agents

The keratolytic agents include salicylic acid, alcloxa, allantoin, glycolic acid and combinations thereof.

The composition of the present invention is preferably prepared in the form of a cream, a gel or a lotion, which contains lubricants, such as glycerol, water soluble celluloses, polysorbates, carbomers, polyethylene glycols (PEG), polyethylene, and thickening agent.

Water Soluble Celluloses

Celluloses are organic compounds with the general formula (C₆H₁₀O₅)ₙ, a polysaccharide consisting of a linear chain of several hundred to over ten thousands β(1→4) linked D-glucose units. Preferred celluloses include water-soluble celluloses, and modified water-soluble celluloses such as those known in the art and have properties similar to cellulose. Examples are methylcellulose of different viscosity, ethylcellulose, hydroxypropyl cellulose, hydroxymethylcellulose, and hydroxyethylcellulose, hydroxypropyl methylcellulose, methocel® MC, and carboxymethylcellulose. These cellulose compounds, like cellulose itself, are not digestible by humans, and they are not toxic, and not allergenic.

Polysorbates

Polysorbates are a class of emulsifiers used in some pharmaceuticals and food preparation. Polysorbates are oily liquids derived from PEG-ylated sorbitan (a derivative of sorbitol) esterified with fatty acids. Polysorbates include but are not limited to polyoxyethylene (20) sorbitan monolaurate (polysorbate 20), polyoxyethylene (20) sorbitan monopalmitate (polysorbate 40), polyoxyethylene (20) sorbitan monostearate (polysorban 60), polyoxyethylene (20) sorbitan tristrearate (polysorban 65), and polyoxyethylene (20) sorbitan monooleate (polysorban 80).

Carbomers

Carbomer is a generic name for synthetic polymers of acrylic acid used as emulsion stabilizers or thickening agents in pharmaceuticals and cosmetic products. They may be homopolymers of acrylic acid, crosslinked with an allyl other pentaerythritol, allyl ether of sucrose, or allyl ether of propylene. Carbomers include but are not limited to carbopol(s) polymer 71G NF, carbopol® polymer 971P NE, carbopol® polymer 974P NF, carbopol® polymer 980 NE, carbopol® polymer 981 NF, carbopol® polymer 5984 EP and carbopol® polymer Ultrez 10 NF.

Polyethylene Glycol (PEG)

PEG refers to an oligomer or polymer of ethylene oxide and are prepared by polymerization of ethylene oxide and are commercially available over a wide range of molecular weights from 300 g/mol to 10,000,000 g/mol. The preferred PEG to be used in the present invention are liquid PEGs including but not limited to PEG 200, PEG 200E, PEG 300, PEG 300E, PEG 400, PEG 400E, PEG 600 and PEG 600E.

Thickening Agents

Thickening agents are often used in cosmetics and personal hygiene products. They include but are not limited to alginic acid, sodium alginate, potassium alginate, ammonium alginate, calcium alginate, agar, carrageenan, locust bean gum, xanthan gum, pectin, and gelatin.

Other Components

The composition of the present invention may also be prepared by the addition of a vasoactive dilator compound, which include but are not limited to niacin, nicotinic acid, visnadine, an icarin compound, amentoflavone, and forskolin. The icarin compound may be chosen from 7-hydroxyethyl-icarin, 7-aminoethyl-icarin, 7-hydroxyethyl-3-0-ramnosyl-icarin, 7-aminoethyl-7-desgluco-3-ramnosyl-icarin, 8-dihydro-icarin and its glucosides in 7 and 3, and 7-hydroxyethyl-7-desgluco-icarin.

The composition of the present invention may also be prepared by the addition of an antiseptic agent in order to keep the composition sterile and disinfect the surfaces onto which it is applied during use. The preferred antiseptic agents include but are not limited to chlorhexidine gluconate, glucono delta-lactone, a paraben compound, benzoic acid, imidazolidinyl urea, a quaternary ammonium compound, and Octenidine dihydrochloride.

The composition of the present invention may also be prepared by the addition of an absorption enhancer. The preferred absorption enhancers include but are not limited to from triglycerides of coconut oil, isopropyl palmitate, isopropyl myristate, laurocapram, glycerol, propylene glycol and derivatives thereof.

Furthermore, in order to stabilize and keep the composition for extended periods of time, preservative agents may be added to the composition. The preferred preservative agents include but are not limited to EDTA, EGTA, hydroxytoluene butoxide, hydroxyanisol butoxide, sodium hydroxide, calcium propionate, sodium nitrate, sodium nitrite, sulfur dioxide, sodium bisulfite, benzoic acid, caprylyl glycol, Diazolidinyl urea, Phenoxyethanol, Dehydroacetic acid, Iodopropynylbutylcarbamate, Sorbic acid, Isopropyl-paraben, Isobutyl-paraben, Butyl-paraben, and potassium hydrogen sulfite.

The composition of the present invention may also be supplemented with a small quantity of fragrance or perfume.

### Alternative Embodiments

### EXAMPLE I

The compound of the present invention may be added to pharmaceutical compositions in approximately 0.25% to 0.75%, to a maximum of 1%.

| Component | Quantity g | |
|---|---|---|
| (2-nitrooxy)-2-ethylamino-3-methylbutanoate | 0.25-0.75 | g |
| hydrochloride | | |
| Capsaicin | 2 to 5 | g |
| Polyethylene glycol 400 | 10.00 | g |
| Ethoxydiglycol (Transcutol - Gattefossė) | 10.00 | g |
| Sorbitol | 10.00 | g |
| Polysorbate 20 | 8.00 | g |
| Carbomer | 1.00 | g |
| Imidazolidinyl urea | 0.30 | g |
| Xanthane gum | 0.30 | g |
| Methyl paraben | 0.20 | g |
| Disodium EDTA | 0.10 | g |
| Hydroxytoluene butoxide | 0.05 | g |
| 10% sol. sodium hydroxide | 2.00 | g |
| Isopropyl myristate | 2.00 | g |
| Perfume | 0.01 | g |
| Water | q.s. to 100 | g |

### EXAMPLE II

| Component | Quantity g | |
|---|---|---|
| (2-nitrooxy)-2-ethylamino-3-methylbutanoate | 0.25-0.75 | g |
| hydrochloride | | |
| Methol | 2-5% | |
| Eucalyptol | 2-5 | g |
| Ethyl ximeninate | 2.00 | g |
| Coleus purified extract >80% | 0.20 | g |
| polyethylene glycol 400 | 10.00 | g |
| Ethoxydiglycol (Transcutol - Gattefossė) | 10.00 | g |
| Sorbitol | 10.00 | g |
| Polysorbate 20 | 8.00 | g |
| Carbomer | 1.00 | g |
| Imidazolidinyl urea | 0.30 | g |
| Xanthane gum | 0.30 | g |
| Methyl paraben | 0.20 | g |
| Disodium EDTA | 0.10 | g |
| Hydroxytoluene butoxide | 0.05 | g |
| Isopropyl palmitate | 1.00 | g |
| 10% sol. sodium hydroxide | 2.00 | g |
| Perfume | 0.01 | g |
| Water | q.s. to 100 | g |

The embodiments and examples presented herein are illustrative of the general nature of the subject matter claimed and are not limiting. It will be understood by those skilled in the art how these embodiments can be readily modified and/or adapted for various applications and in various ways without departing from the spirit and scope of the subject matter disclosed claimed. The claims hereof are to be understood to include without limitation all alternative embodiments and equivalents of the subject matter hereof. Phrases, words and terms employed herein are illustrative and are not limiting. Where permissible by law, all references cited herein are incorporated by reference in their entirety. It will be appreciated that any aspects of the different embodiments disclosed herein may be combined in a range of possible alternative embodiments, and alternative combinations of features, all of which varied combinations of features are to be understood to form a part of the subject matter claimed.

1-82. (canceled)

83. A topical composition comprising:
an effective amount of a compound of formula (I): 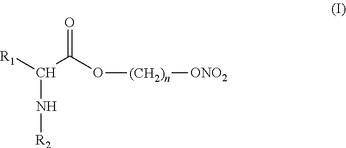
wherein
when n=1-2 and 7-10;
R₁ is an amino acid side chain group (D or L configuration) chosen from: 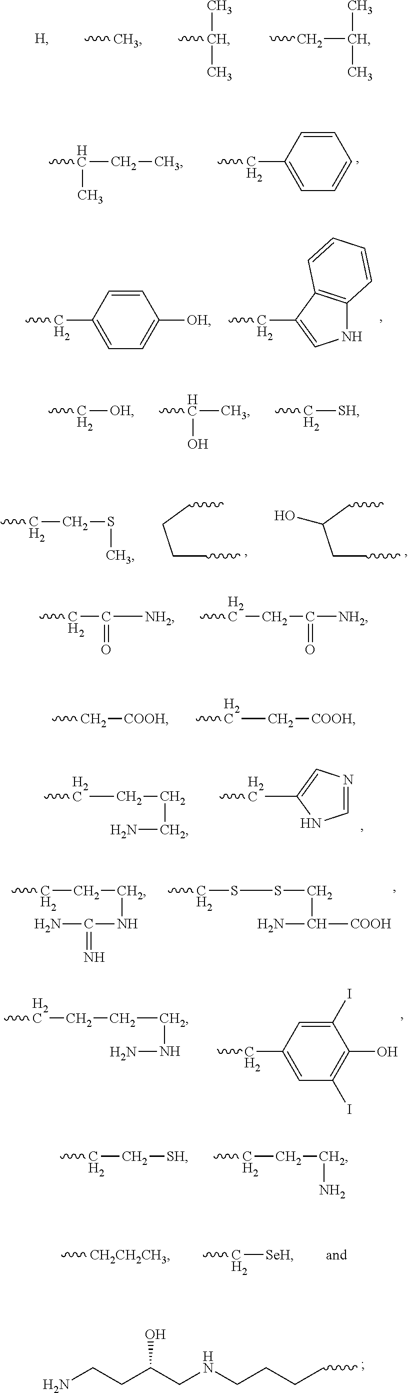
when n=3 to 6;
R₁=an amino acid side chain group (D or L configuration) chosen from: 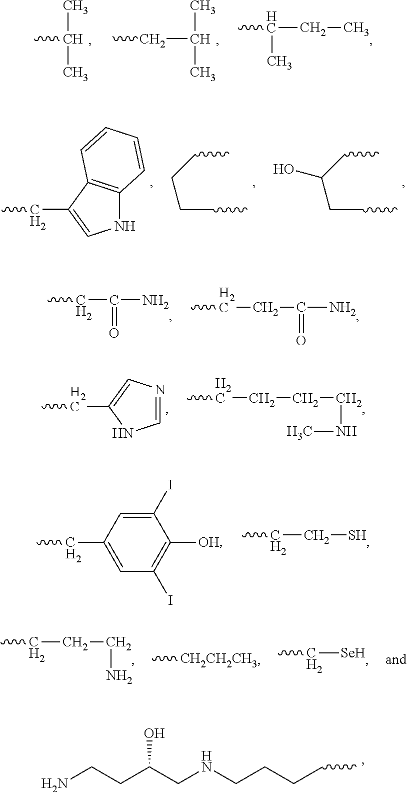
wherein when R₁ is 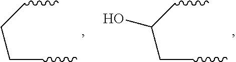
said R₁ is also linked to an NH₂ of said Formula (I) to form a proline or hydroxyproline amino acid side chain,
wherein R₂ is a hydrogen atom, or an amino acid (D or L configuration) forming a peptide bond, or any pharmaceutically acceptable salts thereof; and
an effective amount of a topical analgesic compound topical analgesic compound chosen from a capsaicinoid, resiniferatoxin, cinnamaldehyde, menthol, eucalyptol, camphor, and norcamphor,
in association with a pharmaceutically acceptable topical carrier.

84. The composition as claimed in claim 83, wherein said compound of formula (I) is (2-nitrooxy)-2-ethylamino-3-methylbutanoate: 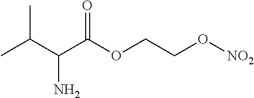
or any pharmaceutically acceptable salts thereof.

85. The composition as claimed in claim 83, wherein said compound of formula (I) is 2′-nitrooxyethyl 2-amino-pentanoate: 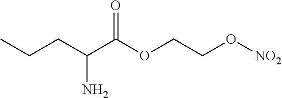
or any pharmaceutically acceptable salts thereof.

86. The composition as claimed in claim 83, wherein said R₂ is a hydrogen atom.

87. The composition as claimed in claim 83, wherein said capsaicinoid is chosen from capsaicin, dihydrocapsaicin, nordihydrocapsaicin, homodihydro capsaicin, homocapsaicin, and nonivamide.

88. The composition as claimed in claim 83, wherein said pharmaceutically acceptable topical carrier is chosen from a water base or an oil base carrier.

89. The composition as claimed in claim 87, wherein said composition comprises a healing promoting agent selected from the group consisting of collagen hydrolysate, aldioxa, hyaluronic acid, elastin, ascorbyl palmitate and combinations thereof.

90. The composition as claimed in claim 87, wherein said composition comprises a dermal circulation enhancer selected from the group consisting of gingko biloba, ginger, ethyl alcohol, arginine, cayenne and combinations thereof.

91. A method of reducing or alleviating pain in a patient which comprises:
(a) topically treating said patient with the composition as claimed in claim 83.

92. A method of reducing or alleviating pain in a patient which comprises:
(a) topically treating said patient with a composition comprising:
an effective amount of a compound of formula (I): 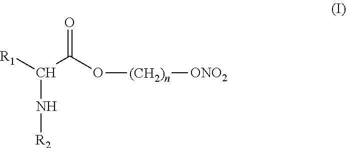
wherein
when n=1-2 and 7-10;
R₁ is an amino acid side chain group (D or L configuration) chosen from: 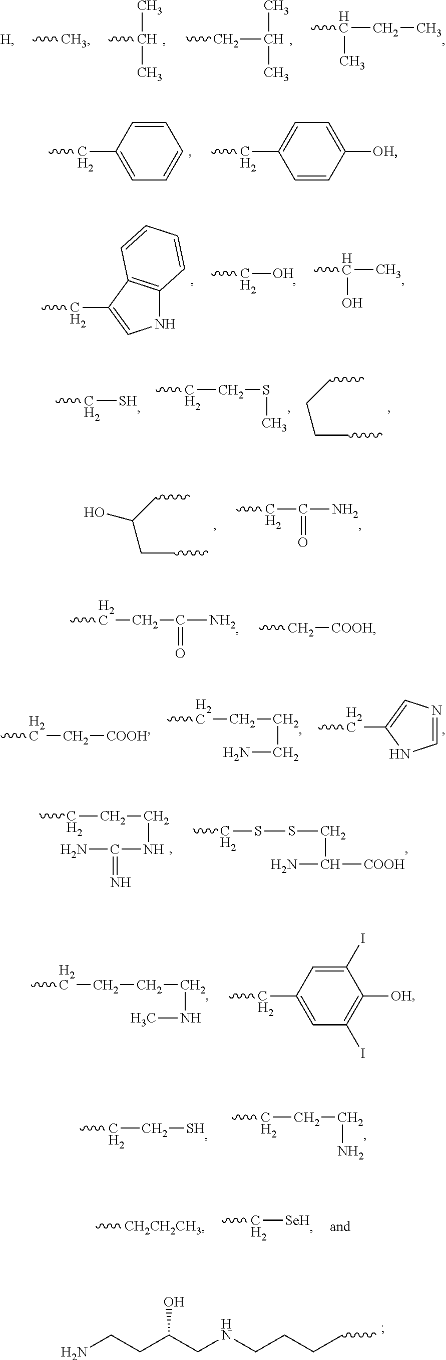
when n=3 to 6;
R₁=an amino acid side chain group (D or L configuration) chosen from: 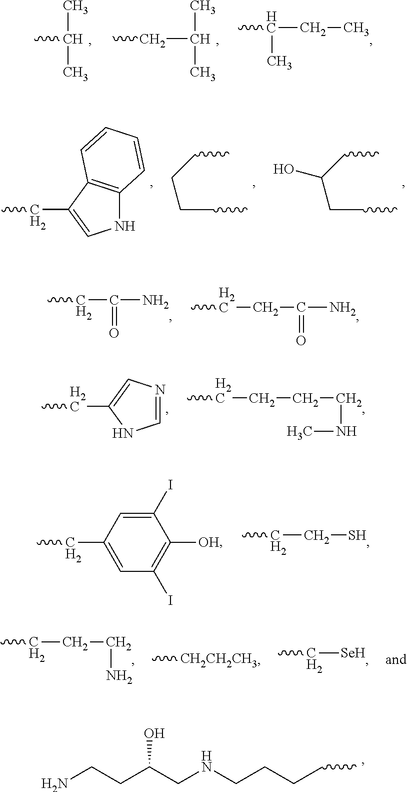
wherein when R₁ is 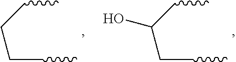
said R₁ is also linked to an NH₂ of said Formula (I) to form a proline or hydroxyproline amino acid side chain,
wherein R₂ is a hydrogen atom, or an amino acid (D or L configuration) forming a peptide bond, or any pharmaceutically acceptable salts thereof; in association with a pharmaceutically acceptable topical carrier.

93. The method as claimed in claim 92, wherein said compound of formula (I) is (2-nitrooxy)-2-ethylamino-3-methylbutanoate: 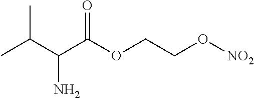
or any pharmaceutically acceptable salts thereof.

94. The method as claimed in claim 92, wherein said compound of formula (I) is 2′-nitrooxyethyl 2-amino-pentanoate: 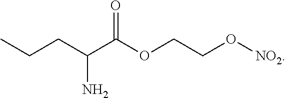
or any pharmaceutically acceptable salts thereof.

95. The method as claimed in claim 92 wherein said R₂ is a hydrogen atom.

96. The method as claimed in claim 92, wherein said pharmaceutically acceptable topical carrier is chosen from a cream, a gel and a lotion.

97. The method as claimed in claim 92, wherein said topical composition comprises a healing promoting agent selected from the group consisting of collagen hydrolysate, aldioxa, hyaluronic acid, elastin, ascorbyl palmitate and combinations thereof.

98. The method as claimed in claim 92, wherein said topical composition comprises a dermal circulation enhancer selected from the group consisting of gingko biloba, ginger, ethyl alcohol, arginine, cayenne and combinations thereof.

99. The method as claimed in claim 91, wherein said chronic pain is associated with at least one of nocturnal muscle cramps, arthritis, rheumatoid arthritis, cancer, chronic neuropathic pain syndromes such as postherpetic neuralgia and painful diabetic neuropathy, fibromuscular diseases, tension, headache, backache, osteoarthritis, migraine, tension headache, anal fissure pain, and Raynaud's Phenomenon.

100. The method as claimed in claim 91, wherein said patient has a normotensive blood pressure, a hypertensive blood pressure, or a hypotensive blood pressure.

101. The method as claimed in claim 100, wherein when blood pressure is a normotensive blood pressure or a hypotensive blood pressure, said topically treating said patient results in a stable blood pressure.

102. The method as claimed in claim 100, wherein when said blood pressure is a hypertensive blood pressure, said topically treating said patient results in a decreased blood pressure that is a normotensive blood pressure.
